(19) United States
(12) Plant Patent Application Publication
(10) Pub. No.: US 2005/0278815 P1
(43) Pub. Date: Dec. 15, 2005

(54) Salvia Greggii Teresa

(76) Inventors: David Thomas Steinbrunner, Hunt, TX (US); Teresa Steinbrunner, Ingram, TX (US)

Correspondence Address: DAVID THOMAS STEINBRUNNER, P.O. BOX 877, HUNT, TX, 78024, US

(21) Appl. No.: 11/059,802

(22) Filed: Aug. 15, 2005

Related U.S. Application Data
(60) Provisional application No. 60/547,316, filed on Feb. 24, 2004.

(51) Int. Cl. A01H 5/00

(52) U.S. Cl. PLT/259

(57) ABSTRACT

The invention Salvia greggii Teresa has a flower unique to the species, Salvia greggii, in that the lower extended petal of the flower has three (3) distinct pink stripes on the white lower petal. No other varieties of Salvia greggii have this distinct characteristic.

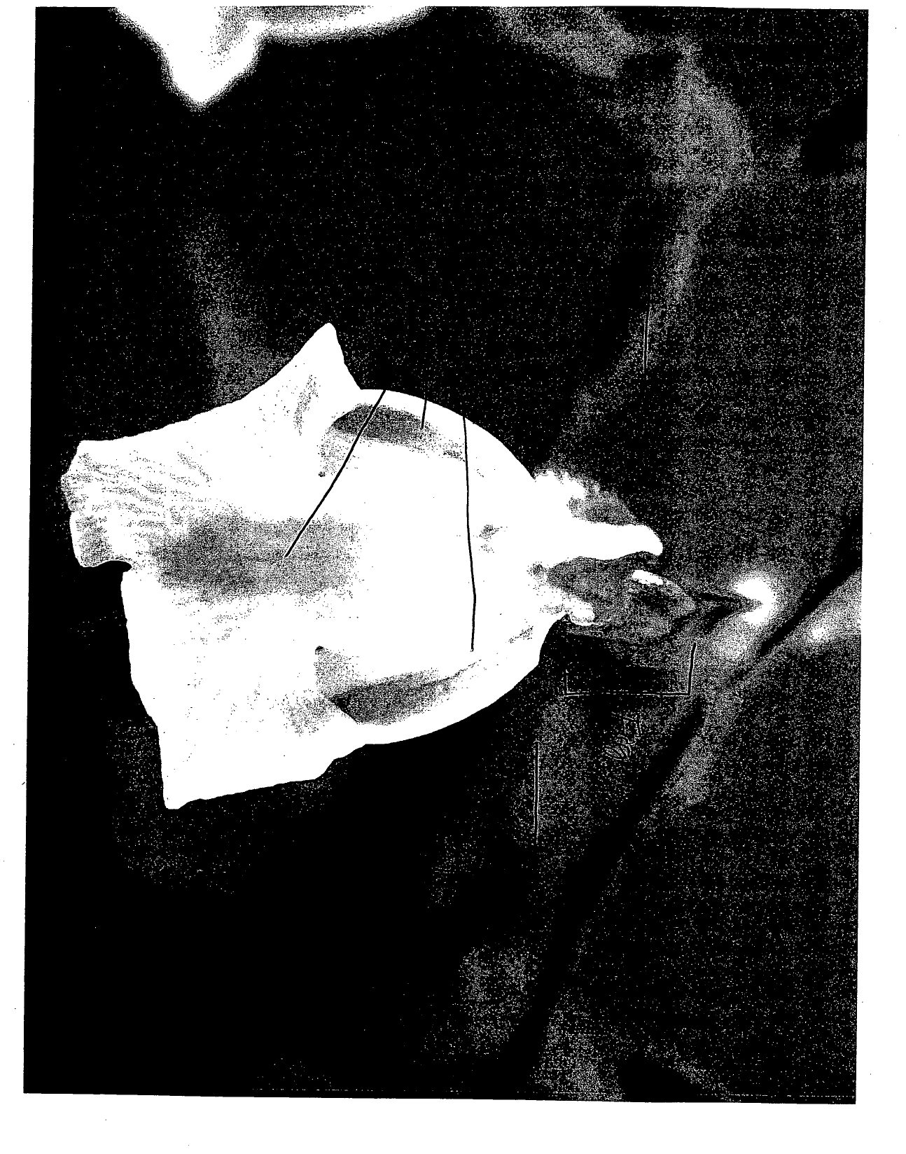

### CROSS-REFERENCE TO RELATED APPLICATIONS

Not applicable

### STATEMENT REGARDING FEDERALLY SPONSORED RESEARCH OR DEVELOPMENT

Not applicable

### REFERENCE TO SEQUENCE LISTING, A TABLE, OR A COMPUTER PROGRAM LISTING COMPACT DISC APPENDIX

Not applicable

### BACKGROUND OF THE INVENTION

Salvia greggii Teresa pertains to the field of plants and is a new invention of the U.S. Patent classification; plants.

Specific documents referring to the invention include ‘San Antonio Express’ newspaper; ‘Los Angeles Times’ newspaper; ‘Native Plant Society of Texas’ newsletter; ‘California Nurserymans Associations’ magazine.

The flowers of Salvia greggii's come in many shades of red, pink, white and blends of the colors. The flower of Salvia greggii Teresa is white with three distinct pink stripes on the lower lip of the corolla. No similar art in the species exists.

### BRIEF SUMMARY OF THE INVENTION

Salvia greggii Teresa gives a new look to the species of Salvia greggii. No known previous art has the distinct coloration of the flower. The invention has been under cultivation since September 1, 2000. The invention was introduced to the market place in March of 2004 via internet sales from www.davesgarden.com and from a contract grower ‘Schumacher's Hillcountry Gardens’ in Newbraunfels, Tex.

### BRIEF DESCRIPTION OF THE SEVERAL VIEWS OF THE DRAWING

Figure A. Figure A depicts thee corolla of the flower of Salvia greggii Teresa which consists of the upper and lower petals of the corolla.

Figure B. Figure B depicts the lower lip (petal) of the petal that has the main interest of the invention, the three pink stripes.

Figure C. Figure C. depicts the three stripes unique to the invention and this art. Two lateral stripes and one mid stripe.

Figure D. Figure D depicts the upper lip (petal) of the invention.

### DETAILED DESCRIPTION OF THE INVENTION

Salvia greggii Teresa is a flower mutation of the species Salvia greggii. Numerous green stem cuttings of Salvia greggii Teresa were taken from a single plant that was under cultivation at the inventors nursery in Hunt, Tex. Each cutting was dipped in a rooting hormone and placed under a mist system rooted in approximately 10 days. As the cuttings grew subsequent cuttings were taken with the same process.

The invention pertains only to the flower and is typical of the species in growth habit, leaf color,texture, stems and hardiness and all vegetative parts of the plant. The invention has remained true to type and after thousands of cuttings has retained all the distinguishing characteristcs of the invention.

The new invention Salvia greggii Teresa pertains only to the coloration of the lower parts. The corolla of the flower has a upper lip (petal) and a lower extended lower lip (petal). The lower lip of the corolla is white with three (3) distinct pink stripes. All three stripes run longitudinally on the the lower lip of the corolla with one pink stripe on the mid axis of the corolla and one stripe on either side running parallel to the mid stripe. The mid stripe runs approximately 90 percent of the lower lip while the two side stripes run approximately 50 percent of the length of the lower lip. The stripes are prevalent from bud break to petal fall, throughout the blooming season.

The mutation was found on only one branch of a red flowering Salvia greggii. The initial cuttings have retained their distinguishing characteristic for four and a half (4.5) years. It is the intention of the inventor to continue marketing the invention and introducing the invention to the plant nursery industry.

1. The inventors of the the invention Salvia greggii Teresa claim the three (3) distinct stripes of the white lower lip (petal) of the the corolla (petals) are a new art.
